(19) United States
(12) Patent Application Publication
(10) Pub. No.: US 2026/0134997 A1
(43) Pub. Date: May 14, 2026

(54) Order-based Diagnosis of Cervical Cancers

(71) Applicants: Board of Trustees of Western Michigan University, Kalamazoo, MI (US); University of Pittsburgh - Of The Commonwealth System Of Higher Education, Pittsburgh, PA (US); Universit of Hawaii, Honolulu, HI (US)

(72) Inventors: Robert Allen Makin, III, Mattawan, MI (US); Steven Durbin, Honolulu, HI (US); Jennifer Makin, Pittsburgh, PA (US)

(21) Appl. No.: 19/386,425

(22) Filed: Nov. 12, 2025

Related U.S. Application Data
(60) Provisional application No. 63/719,633, filed on Nov. 12, 2024.

(51) Int. Cl. G16H 50/20; G06T 7/00; G16H 30/40; G16H 50/70

(52) U.S. Cl. CPC G16H 50/20; G06T 7/0014; G16H 30/40; G16H 50/70; G06T 2207/10056; G06T 2207/30096

(57) ABSTRACT

Aspects described herein may include a method of diagnosing cervical cancer and pre-cancer conditions, including: utilizing a computer to extract a numerical value corresponding to an order parameter squared (S²) from image data from at least one image of cervical tissue/cells from a patient, wherein the image data includes at least one region having light regions and dark regions, and wherein S² includes a numerical value quantifying a degree of order present in the image data, and wherein the extracted numerical value comprises a ratio of an area of the light regions to a total area; comparing the extracted numerical value to one or more predefined diagnosis ranges corresponding to stages of cervical cancer and pre-cancer conditions; and diagnosing the stage of the cervical cancer and pre-cancer conditions if the numerical value of the S² falls within one of the predefined diagnosis ranges.

### CROSS-REFERENCE TO RELATED APPLICATION

The present application claims priority to U.S. Provisional Application 63/719,633, filed Nov. 12, 2024, the contents of which are included by reference in their entirety.

### NAMES OF PARTIES TO A JOINT RESEARCH AGREEMENT

The presently claimed invention was made by or on behalf of the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are the Board Of Trustees Of Western Michigan University, the University Of Pittsburgh—Of The Commonwealth System Of Higher Education, and the University Of Hawaii.

### BACKGROUND TO THE INVENTION

Despite being a preventable illness, cervical cancer remains the fourth most common malignancy among women, with an incidence of over 600,000 cases per year (Wang et al. 2024). The oncogenesis of human papillomavirus (HPV) and the pathophysiology of pre-cancerous lesions, cervical intraepithelial neoplasia grades 2 and 3 (CIN2/3) are well known (Loopik et al. 2021). The World Health Organization has called for action to eliminate cervical cancer by 2030 through increased vaccination against HPV as well as increased access to cervical cancer screening and the treatment of cervical disease (Confell, 2019). Traditional screening with cytology in Western countries has led to reductions of mortality rates by up to 70% (Safaeian et al. 2007). However, great disparities exist across resource settings, and cervical cancer rates remain high among the under-screened. For instance, across Africa, cervical cancer was the leading cause of cancer-related death in women in 2018 with a notable increase in cancer incidence and mortality in low-income and developing countries (Buskwofie et al. 2020). According to the World Health Survey, the lowest access to cervical cancer screening was seen among those with low socioeconomic status and rural residence (Akinyemiju et al. 2016). The challenge that remains is how to overcome the barriers of geography, health care infrastructure, and staffing shortages to provide equal and effective care globally.

Current cervical cancer prevention models of care depend on location and available resources, but usually involve multiple visits and pathologic review of two to three specimens. Automated detection and classification of cervical precancerous lesions is a rapidly progressing field, one which promises to reduce barriers inherent to healthcare. Methods of the prior art have included cell segmentation, automated cytology screening systems and deep learning classifier models (see, e.g., Hou et al. 2022; Sehra and Bhatla, 2023). These methods suffer from challenges based on single-cell identification, are sensitive to image quality, and may be limited in their application to either cytology, colposcopy, or histology.

While traditional approaches have improved outcomes, the integration of innovative physical science techniques could help to simplify screening for the complex mechanism of HPV carcinogenesis in squamous cervical cells and enhance diagnostic accuracy. In the 1930s, Bragg and Williams (1934) characterized structural ordering in crystalline lattice systems with a single numerical value, the Bragg-Williams order parameter(S). It has been shown theoretically and confirmed experimentally that the squared version of this parameter (S²) has a direct linear relationship to system-level properties and can be used to predict key properties of various physical systems, including biological cells (Laks et al. 1992; Makin et al., 2019, 2020). The Bragg-Williams measure of structural ordering can be adapted to measure the degree of ordering in other physical systems, such as polymers or organic systems (Id.). It is possible to extend the concept of S to cellular systems. Underlying the methodology is the concept of structural motifs, which describe the nearest neighbor environment depending on the resolution of a particular image (e.g., proteins with electron microscopy techniques, or cellular, multicellular, and/or tissue-level structures with images at lower magnifications). These order parameters can be used to distinguish benign, pre-cancerous and malignant conditions using a numerical value of an order parameter (S²) extracted from cellular images using a numerical analysis.

By applying this concept to cervical cells, we can utilize numerical analysis to extract S² values from cellular images, providing a quantitative measure to differentiate between benign, pre-cancerous, and malignant conditions. This novel approach could complement existing screening methods, potentially improving early detection and treatment outcomes for cervical cancer.

Further, as noted in the Examples below, another advantage of the systems and methods are not limited to certain length scales, but can range from cytology images at the cellular and tissue level, to histopathological images, to colposcopy images (i.e. images at the tissue level, e.g, of the cervical surface).

### SUMMARY OF THE INVENTION

Some aspects of the disclosure may include a computer-implemented method of diagnosing cervical cancer and pre cancer conditions in a patient utilizing an order parameter, the method including: utilizing a computer to extract a numerical value corresponding to an order parameter squared (S²) from image data corresponding to at least one image of cervical tissue or cells from a patient, wherein the image data includes at least one region having light regions and dark regions, and wherein S² includes a numerical value quantifying a degree of order present in the image data, and wherein the extracted numerical value comprises a ratio of an area of the light regions to a total area that is equal to the sum of an area of the light regions and an area of the dark regions; comparing the extracted numerical value to one or more predefined diagnosis ranges corresponding to stages of cervical cancer and pre cancer conditions; and diagnosing the stage of the cervical cancer and precancer conditions if the numerical value of the extracted S² falls within one of the predefined diagnosis ranges.

In others, the extracted numerical value may include an extracted S² value; and including: determining that the stage of the cervical cancer and precancer conditions is likely to be benign if the extracted S² value is greater than a predefined benign S² value, wherein the predefined benign S² value is the highest of one or more diagnosis ranges corresponding to stages of cervical cancer and pre cancer conditions.

In yet other aspects, the method may further include determining that the stage of cervical cancer and precancer conditions is likely to be a precancer condition if the extracted S² value is within a predefined precancer S² value range, wherein the predefined precancer range is a value between the predefined benign S² range and the highest S² value of one or more diagnosis ranges corresponding to stages of cervical cancer.

In others, the method may include determining that the stage of cervical cancer and precancer conditions is likely to be a cervical cancer stage if the extracted S² value is below the predefined precancer S² value range. In yet others, the one or more predefined diagnosis ranges corresponding to stages of cervical cancer and pre cancer conditions comprise one or more predefined S² value ranges that are determined by utilizing a computer to extract a numerical value of S² from a plurality of sets of data corresponding to images including images of cervical tissue or cells preclassified as one or more stages of cervical cancer and pre cancer conditions.

In other aspects, extracting a numerical value corresponding to S² from image data includes selecting a region of interest that includes at least a portion of at least one image of cervical tissue or cells.

In others, the image data includes at least some tissue or cells from the patient that are free of cervical cancer or precancer conditions; and the region of interest does not include a portion of the least one image of cervical tissue or cells that is suspected to contain cervical cancer or pre cancer conditions.

In yet others, the image data comprises a plurality of pixels; and extracting a numerical value corresponding to S² from the image data includes utilizing a computer to create a pixel intensity histogram of the region of interest.

In other aspects, extracting a numerical value corresponding to S² from the image data includes utilizing a computer to fit first and second curves to the pixel intensity histogram corresponding to the light and dark regions, respectively. In others, extracting a numerical value corresponding to S² from image data includes utilizing a computer to convert the image data to greyscale image data; utilizing a computer to determine a threshold value of the pixel intensity between peaks of the first and second curves; and utilizing a computer to perform a binary threshold on the region of interest using the threshold value to form a set of digital data corresponding to an image having only black and white pixels.

In others, the image data may be a selected one of digital optical image data or image data from a microscope. In yet other aspects, selecting a region of interest comprises utilizing a computer to determine a border using an algorithm.

Other aspects of the present disclosure may include a computer-implemented method of distinguishing between benign and malignant conditions utilizing a numerical value determined from data corresponding to one or more images of cervical cancer or precancer cells or tissue, the method including: utilizing a computer to extract a numerical value from image data corresponding to a digital image of cervical cancer or precancer cells or tissue, wherein the extracted numerical value comprises an extracted numerical value of an order parameter squared (S²), and wherein the digital image corresponding to the image data includes at least one region of concern comprising a potential cervical cancer or precancer condition cell or tissue having a total area including light regions and dark regions, and wherein the numerical value is determined, based at least in part, on an area of a selected one of the light regions and the dark regions to a total area, wherein the total area is equal to the sum of the areas of the light regions and the areas of the dark regions; estimating the likelihood that the potential cervical cancer or precancer cell or tissue is cervical cancer or precancerous condition cell or tissue based, at least in part, on a comparison of the extracted numerical value to one or more predefined numerical criteria that take into account the likelihood that the cell or tissue is cervical cancer or precancerous.

In yet others, the numerical value is determined by dividing the area of the light regions by the total area. In others, the method may include determining a threshold brightness value for the digital image; determining the area of the light regions by summing the areas of the pixels having a brightness value above the threshold brightness value.

In others, the extracted numerical value comprises an extracted numerical value of an order parameter squared (S²). In others, the extracted numerical value comprises an extracted S² value; and includes: determining that the stage of the cervical cancer and precancer conditions is likely to be benign if the extracted S² value is greater than a predefined benign S² value, wherein the predefined benign S² value is the highest of one or more diagnosis ranges corresponding to stages of cervical cancer and pre cancer conditions.

In yet others, the method may further include determining that the stage of cervical cancer and precancer conditions is likely to be a precancer condition if the extracted S² value is within a predefined precancer S² value range, wherein the predefined precancer range is a value between the predefined benign S² range and the highest S² value of one or more diagnosis ranges corresponding to stages of cervical cancer.

In others, the method may further include determining that the stage of cervical cancer and precancer conditions is likely to be a cervical cancer stage if the extracted S² value is below the predefined precancer S² value range.

The methods and systems discussed above and below are not limited to certain length scales, but can range from cytology images at the cellular and tissue level, to histopathological images, to colposcopy images (i.e. images at the tissue level, e.g, of the cervical surface).

Other features and advantages of the present invention will become apparent from the following detailed description, including the drawing. It should be understood, however, that the detailed description and the specific examples, while indicating preferred embodiments, are provided for illustration only, because various changes and modifications within the spirit and scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described herein are for illustrative purposes only of selected embodiments and not all possible implementations and are not intended to limit the scope of the present disclosure.

FIG. 1 shows a flow chart of an example method for calculating the threshold value of an image;

FIG. 2 depicts an example process for finding the threshold value from the fitted curves;

FIG. 3A depicts a schematic of a method of calculating an order parameter using calculation of four total skewed Gaussian distributions in an example aspect of the present disclosure;

FIG. 3B depicts a schematic of a method of calculating an order parameter using calculation of three total skewed Gaussian distributions in an example aspect of the present disclosure;

FIG. 4 depicts pixel intensity histograms for three representative disease classifications: (a) intermediate squamous cell, (b) moderately dysplastic cell; (c) severely dysplastic cell;

FIG. 5 depicts pixel intensity histograms for all disease classes: normal (a) metaplastic cell, (b) intermediate squamous, (c) parabasal, (d) superficial squamous, (e) superficial intermediate; low grade squamous intraepithelial lesion (LSIL) (f) koilocytotic, (g) mild dysplasia; and high grade squamous intraepithelial lesion (HSIL) (i) moderate dysplasia, (j) severe dysplasia, and (k) carcinoma in situ;

FIG. 6 shows a STARD flow diagram for the order parameter analysis index test using the images from the Herlev and SiPaKMeD databases, where n is the number of subjects at each stage;

FIG. 7 displays representative cells from each classification within the Herlev and SIPaKMeD databases, with their associated heat maps;

FIG. 8 displays a regression analysis of the relationship between observed S² values from colposcopy images and the confirmed histology diagnoses from the IARC database;

FIG. 9 shows increased ordering over time upon treatment with a topical therapy for a patient with high-risk HPV and a low-grade squamous intraepithelial lesion;

FIG. 10 displays the robustness of order computations to artificial introduction of different types of noise into cell images, with original cell image at center.

### DETAILED DESCRIPTION OF THE INVENTION

The Bragg-Williams method originally was developed to measure the ordering of the arrangement of atoms in lattice materials using the intensities of peaks in x-ray diffraction patterns; the modified approach extends this framework to non-lattice-based physical systems using wave-based imaging techniques. This new capability enables the measurement of the degree of structural entropy in biological systems spanning length scales from cells to organs. Additionally, this provides a quantitative means to study the relationships and correlations between the degree of structural entropy and properties and mechanisms of biological systems.

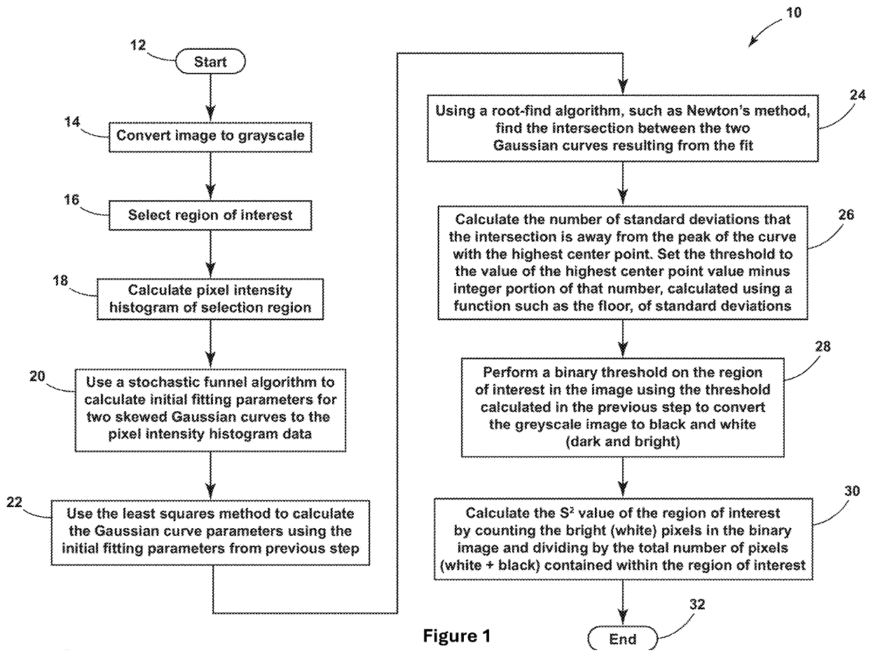

An example illustrative process is shown in FIG. 1, which shows a flow chart of an example process 10 for calculating the threshold value of an image 10. One or more steps of process 10 may be implemented utilizing a computer. For example, a computer may be configured (programmed) to execute one or more of the steps of process 10. Process 10 starts as shown at 12, and step 14 includes converting the image to greyscale. A region of interest may be selected as shown in step 16. In general, the region of interest may be selected before or after converting an image to greyscale. The region of interest may be selected by an individual inspecting one or more images of suspected cervical cancer or precancerous tissue or cells, or the region of interest may be selected by a computer algorithm. The region of interest may have virtually any shape as required to bound a representative portion (region) of the tissue or cells of interest.

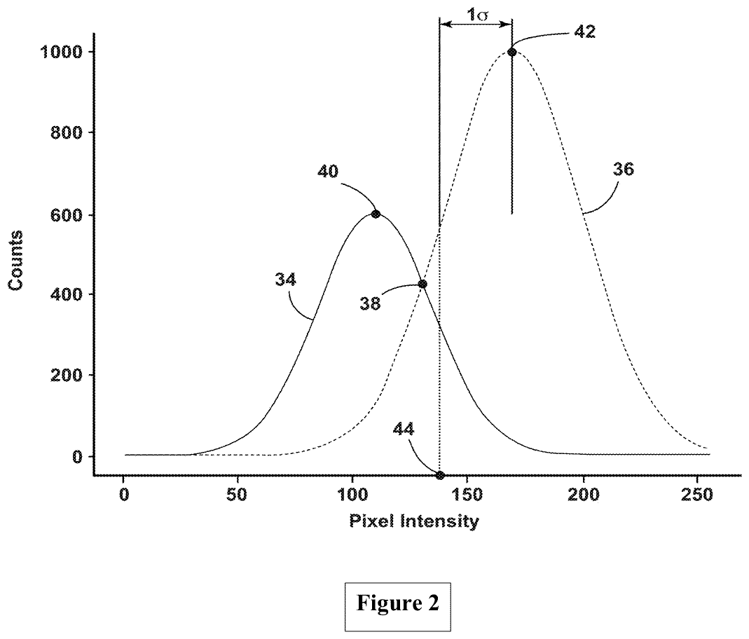

The process 10 further includes calculating a pixel intensity histogram of the selected region. At step 20, an algorithm (e.g., a stochastic funnel algorithm) is used to calculate initial fitting parameters for two skewed Gaussian curves to the pixel intensity (brightness) histogram data. Referring again to FIG. 2, at step 22, the least squares method may be used to calculate the Gaussian curve parameters using the initial fitting parameters from step 20.

At step 24, a root-finding algorithm (e.g., Newton's method) is used to find the intersection between the two Gaussian curves resulting from the curve fit. At step 26, a number of standard deviations that the intersection is away from the curve where the highest center point is calculated. A threshold value is set to the value of the highest center point value minus the floor of that number of standard deviations. As discussed below, the threshold value may be used to determine which pixels are “bright” (white), and which pixels are “dark” (black).

At step 28, a binary threshold is performed on the region of interest in the image using the threshold calculated in step 26. Pixels having an intensity that is greater than the threshold value are given (assigned) a white (high) intensity value, and pixels having an intensity that is less than the threshold value are given (assigned) a black (low) intensity value. In general, the result of the binary threshold is a black (dark) and white (bright) image (not shown) having white (ordered) regions and black (disordered) regions. At step 30, a numerical value, which may comprise the squared order parameter (S²) value of the region of interest, is calculated by counting the bright (white) pixels in the thresholded image and dividing this number by the total number of pixels contained with the region of interest. The total number of pixels is equal to the sum of the number of dark (black) pixels and the number of bright (white) pixels. Because the sizes (areas) of each of the pixels are the same, the S² value is the ratio of the area of the bright regions to the total area. The method 10 then ends as shown at 32. It will be understood that the numerical value may also comprise the ratio of the area of the dark regions to the total area (i.e. S²).

The numerical value (e.g. S²) determined utilizing the process 10 of FIG. 1 may then be compared to predefined criteria (e.g. S²) values extracted from images known to be malignant or benign in order to estimate the chances that the region of interest includes a malignant condition. For example, a computer may be configured (programmed) to generate a “likely benign” output or signal if the numerical value of S² is greater than a predefined value (e.g., 0.4, 0.5, 0.6), and a computer may be configured (programmed) to generate a “likely malignant” output or signal if the numerical value of S² is less than the predefined value (e.g., 0.4, 0.5, 0.6). Also, a computer may be configured to generate an “indeterminate signal” is the numerical value of S² is between a predefined “likely benign” numerical value (e.g., 0.55) and a predefined “likely malignant” numerical value (e.g., 0.4).

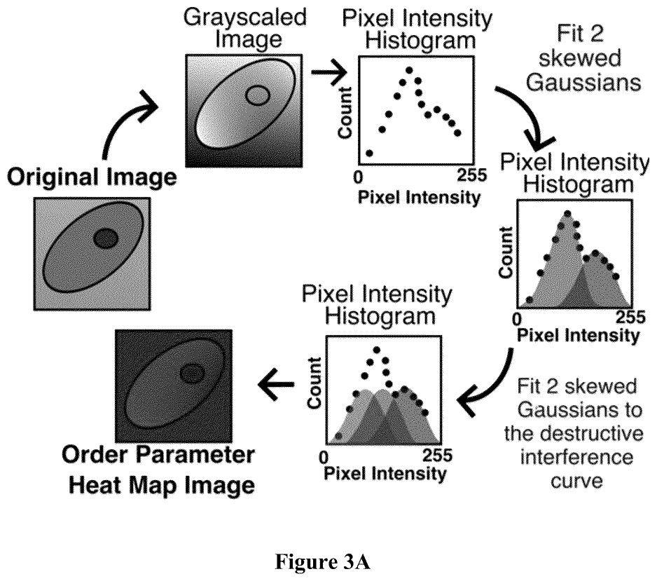

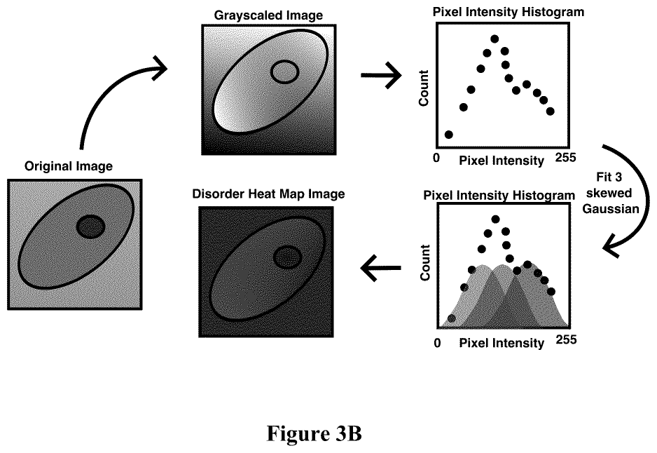

FIG. 2 shows the process for finding the threshold value 44 from the fitted curves 35 and 36. As discussed above in connection with FIG. 1, a disordered curve 34 and ordered curve 36 may be fitted using, for example, skewed Gaussian curved parameters. The inner section 38 of the curves 34 and 36 may then be determined (e.g., using Newton's method), and the threshold value 44 may then be determined. In the example of FIG. 3, the disordered curve 34 has a center point (peak) 40, and the ordered curve 36 has a center point (peak) 42. In the example of FIG. 3, the threshold value 44 is approximately 140. It will be understood that the present disclosure is not limited to a specific technique or approach for determining the threshold value.

Other example processes may be used to calculate S². The S² value of a cell, tissue or organ can be measured from an optical image through analyzing the pixel intensity histogram of the image. For a system with some degree of structural ordering, the probability of a given portion of the system being ordered is P_{order}=S and the probability that a given part of the system is disordered then is P_{disorder}=1−P_{order}=1−S. Regardless of the wavelength, a wave impinging upon the lattice has a probability of S of interacting with an ordered portion of the lattice and a probability of 1−S² of interacting with a disordered portion of the lattice. The probability of a wave originating from an ordered portion will interfere constructively with another wave originating from an ordered site is P_{constructive}=S², where P_{constructive} is the probability of constructive interference occurring between the two waves. For the other two cases—two waves both originating from disordered regions or one from a disordered region and one from an ordered region—the interference will be destructive and thus the probability of destructive interference occurring is P_{destructive}=1−S². Thus, the S² value of a structure can be obtained by measuring the probability of occurrence of constructive interference from waves interacting with the biological sample. From a physical perspective, this probability can be determined by analyzing the pixel intensity histogram of the detected image.

As shown in FIG. 3A, the first step in the analysis is to convert the original color image to grayscale. The pixels are then plotted on a histogram by their pixel intensity and frequency or count. As above for the example discussed in FIGS. 1 and 2, the computer-aided algorithm fits two curves, typically skewed Gaussian, to the pixel intensity histogram using non-linear least squares. The first curve seen at lower pixel intensities is due to light waves destructively interfering at the detector after interacting with disordered regions of the cells. The second curve, seen at higher pixel intensities, corresponds to light waves interfering constructively at the detector after interacting with ordered regions of the cells. Based on the two fitted curves, a threshold value is chosen at an integer multiple of the standard deviation away from the center of the ordered curve, based on relative location of the intersection between the ordered and disordered structure curves. This threshold value is used to separate ordered and disordered contributions to the image; the S² value of the cell is the ratio of the total area of ordered structure regions in the image to the total pixel area of the image.

At the camera sensor, constructive interference will result in higher pixel intensities compared to destructive interference. However, other factors may broaden the range of intensity values in the image, including absorption, surface roughness, and sample geometry. These factors can be accounted for by assuming each is random variable and thus their total effect can be approximated by a Gaussian distribution. Thus, by fitting two Gaussian curves to a pixel intensity histogram using a least squares or similar fitting process, one at higher pixel intensities due to constructive interference and one at lower pixel intensities, the integrated area under the constructive interference curve divided by the sum of the integrated areas under the constructive and destructive interferences curves is equal to S². Additionally, the uncertainty of the measured S² value can be determined by a Monte Carlo approach using the calculated uncertainties of the fitted curves parameters during the integration step.

By further considering the cases of destructive interference, there are two possibilities for this to occur: one where both waves have interacted with disordered portions of the system and the other where one wave has interacted with an ordered portion of the system and the other wave has interacted with a disordered portion of the system. These two cases will also appear as Gaussian curves in the pixel intensity histogram, but they will sum to the total destructive interference Gaussian curve that was fit. The three fitted curves can then be used to calculate expectation values of S for each pixel value. The expectation value of S² for each pixel intensity can then be used to assign S² values to each pixel within the image by their corresponding pixel intensity value, allowing for the creation a mapping of S² values across the entire image. These additional steps are shown in FIG. 3A.

An alternative approach to this method is to fit three Gaussian curves directly to the pixel intensity histogram, instead of first fitting two Gaussians and then fitting two additional Gaussian curves to the disordered curve. In this case, S² can be calculated from the ratio of the area under the curve whose peak is located at the highest pixel intensity to the sum of the area under all three fitted curves. Additionally, the mapping of S² at a pixel level across the image can then be done as in the previous method—by calculating the expectation value of S for each pixel intensity value using the three fitted curves. This three-curve fitting process is shown in FIG. 3B.

The following examples show the sensitivity and specificity of the modified Bragg-Williams structural order parameter, measured through image analysis, for cervical squamous cell dysplasia compared to the gold standard of cytopathology. Secondary objectives included identifying the sources of disorder within the cell, assessing the impact of image quality on the measured order parameter, and determining the impact of image quality on our methodology.

Example 1. We conducted a retrospective cohort study of the SIPaKMeD and Herlev public access databases for cervical cytology. These gold-standard pathologic diagnosis databases previously classified images of cervical squamous cells into categories ranging from normal to dysplastic. The SIPaKMeD database consists of 4,049 images of isolated cells that were manually cropped from images of Papanicolaou slides and distributed over five classifications for cervical cytology (Plissiti et al. 2018). The Herlev database contains 917 images of cells manually cropped from Pap smear slides distributed over seven classifications for cervical cytology (Jantzen and Dounias, 2006). The distribution of the cells in classes for each database is depicted in Table 1.

TABLE 1
Diagnostic Accuracy of Order Parameter-Based Image Analysis Method

| | | Database | | | | | |
|---|---|---|---|---|---|---|---|
| | S² | Si = SIPKaMed; | | S² | | | |
| Cell Type | [min, | H = Herlev, | Bethesda | [min, | | | |
| (n) | max] | (N) | Classif. | max] | Sensitivity | Specificity | AUC |
| Dyskeratotic | [0.6837, | Si | Normal | [0.6837, | 1.0 | 1.0 | 1.0 |
| (813) | 0.7635] | | | 0.8633] | | | |
| Superficial- | [0.7181, | Si | | | | | |
| intermediate | 0.8373] | | | | | | |
| (813) | | | | | | | |
| Koilocytotic | [0.7236, | Si | | | | | |
| (825) | 0.7635] | | | | | | |
| Metaplastic | [0.8233, | Si | | | | | |
| (793) | 0.8633] | | | | | | |
| Parabasal | [0.7629, | Si (787) | | | | | |
| (837) | 0.8228] | H (50) | | | | | |
| Intermediate | [0.7665, | H | | | | | |
| Squamous | 0.8262] | | | | | | |
| (120) | | | | | | | |
| Superficial | [0.7356, | H | | | | | |
| Squamous | 0.7941] | | | | | | |
| (124) | | | | | | | |
| Columnar | [0.7560, | H | | | | | |
| (148) | 0.8358] | | | | | | |
| Mild | [0.5800, | H | LSIL | [0.5800, | 1.0 | 1.0 | 1.0 |
| Dysplasia | 0.6399] | | | 0.6399] | | | |
| (282) | | | | | | | |
| Moderate | [0.5101, | H | HSIL | [0.3289, | 1.0 | 1.0 | 1.0 |
| Dysplasia | 0.5500] | | | 0.5500] | | | |
| (246) | | | | | | | |
| Severe | [0.4332, | H | | | | | |
| Dysplasia | 0.5130] | | | | | | |
| (297) | | | | | | | |
| Carcinoma | [0.3289, | H | | | | | |
| In Situ | 0.3888] | | | | | | |
| (150) | | | | | | | |

In total, 5448 images of cells from the SiPaKMed and Herlev Pap smear databases were analyzed using a modified Bragg-Williams approach to measure the degree of structural ordering in the cells.

Images of cells were blindly interpreted and assigned an order parameter; zero was set as complete disorder and one as perfect order. The results were then sorted by their pathology confirmed diagnosis, which is the reference standard. The SIPaKMeD and Herlev database do not utilize the Bethesda Classification for cells, and for consistency, future use, and replication their classifications were grouped into the three corresponding Bethesda Categories: normal, low grade squamous intraepithelial lesion (LSIL), and high grade squamous intraepithelial lesion (HSIL) (Pangarkar, 2012). The S² ranges for all cytology images that fell within the categories of normal, low grade and high grade were determined.

Using a random selection of 70% of images from both databases, the S² ranges for the three Bethesda classifications were then used to determine threshold S² values to create a multiclass classifier. This model was then run on the remaining 30% of the images to calculate its sensitivity, specificity, and Area Under the Receiver Operating Characteristic Curve (AUC) using a one versus rest approach for each of the classes.

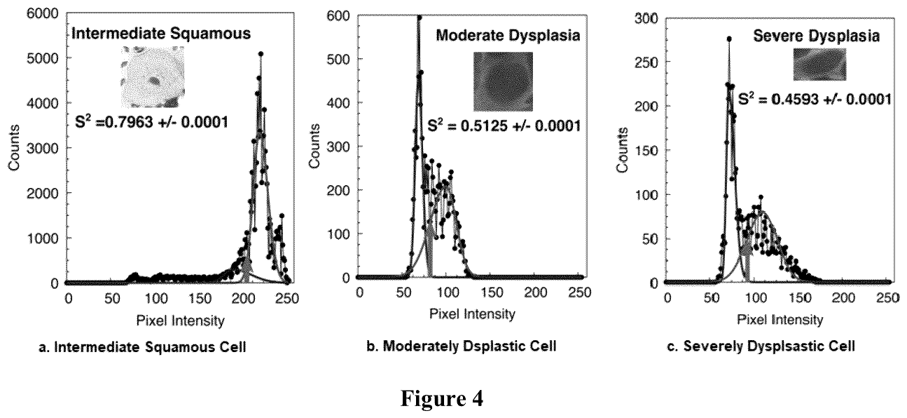

A total of 5,448 images were analyzed using our image analysis program and assigned order parameters. There were no inconclusive test results. Displayed in FIG. 4 are pixel histograms with the associated image for three representative cell classifications from the Herlev database, namely, (a) intermediate squamous cell, (b) moderate dysplasia and (c) carcinoma in situ. In all three histograms, the fitted disordered curve (blue line) represents pixel intensities corresponding to light waves that have interacted with disordered portions of the cell. The fitted ordered curve (red line) represents pixel intensities corresponding to light waves that have interacted with ordered portions of the cell. The green arrow indicates the determined pixel intensity threshold used to calculate S².

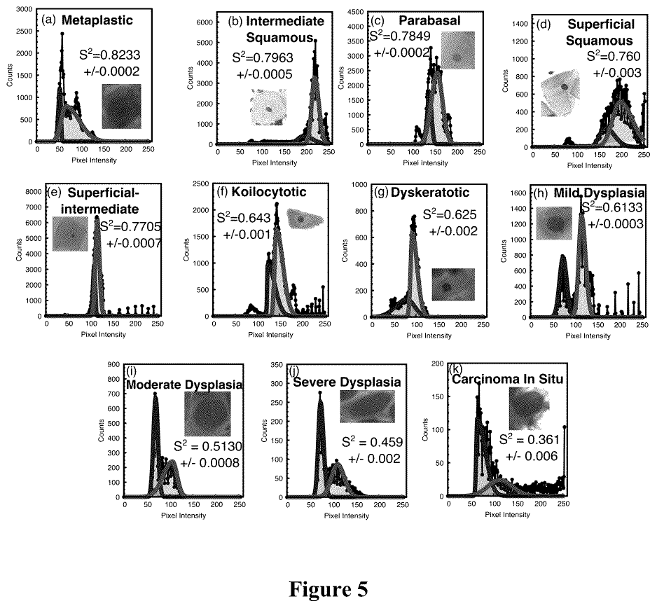

FIG. 5 depicts the fitted pixel histograms used to calculate Se for a representative sample for for each cell type classification in the Herlev and SIPaKMeD databases In the normal cell-type classifications, the S² values were as follows: (a) metaplastic cell 0.8233+/−0.002; (b) intermediate squamous 0.7963+/−0.0005 (c) parabasal 0.7849+/−0.0002, (d) superficial squamous 0.760+/−0.003, and (e) superficial intermediate 0.7705+/−0.0007. For Bethesda classification of low grade squamous intraepithelial cells, the LSIL S² values were: (f) koilocytotic 0.643+/−0001; and (g) mild dysplasia 0.6133+/−0.0003. For HSIL cell classifications, the S² values were: (i) moderate dysplasia 0.5130+/−0.0008; (j) severe dysplasia 0.459+/−0.002; and (k) carcinoma in situ 0.361+/−0.006.

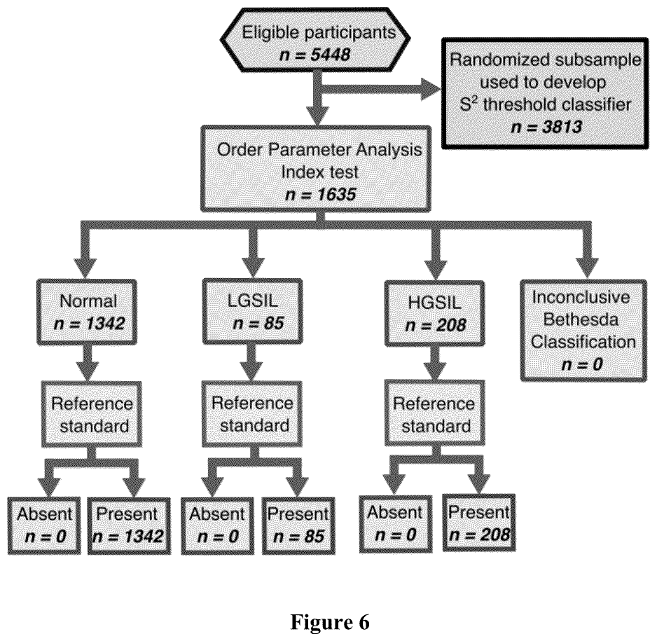

For all images analyzed, the measured degree of order classified normal, LSIL and HSIL cell images with no overlap in ranges (see FIG. 6). Each category fit within well defined, narrow ranges. Between the two databases, 1,342 cytology images were classified as normal with order parameters falling between 0.687 and 0.8633 with a deviation of 0.054. A total of 85 cytology images were classified as LSIL with ranges falling between 0.580 and 0.6399 with a deviation of 0.0599. Finally, 208 cytology images were classified as HSIL with order parameters between 0.3289 and 0.550 with a deviation of 0.2211. Initial results showed a strong trend between S² and the cytopathologic classification, with greater disorder correlating with more severe dysplasia (FIG. 6).

Of note, the Herlev category of moderate dysplasia, which was sorted under HSIL, had a range between 0.5101 and 0.5500, which was closer to that of Herlev cells classified as mild dysplasia (LSIL range 0.5800-0.6399) than to the Herlev classifications of high grade and carcinoma in situ, which had distinctly lower order ranges [0.4332-0.5130 and 0.3289-0.388, respectively] (Table 1).

In the multiclass classifier analysis, 1,590 or 30% of the images were classified by the range category they fell within and then cross-referenced with their pathology diagnosis. For all the images analyzed, the measured degree of order classified cytology images as normal, low grade or high grade with perfect accuracy (Table 1).

Example 2. To examine the spatial relationship of the order parameter and its association with cervical dysplasia, the technique of forming heat maps of the order parameter across histology slide images is applied to locate the origin of disorder at the cellular level. The pixel histogram intensity analysis measurement of S² can be extended to create a mapping of S² across by further considering the cases of destructive interference. There are two possibilities for this to occur: one where both waves have interacted with disordered portions of the system (DD) and the other where one wave has interacted with an ordered portion of the system and the other wave has interacted with a disordered portion of the system (DO). These two cases will also appear as Gaussian curves (f_{DO} and f_{DD}) in the pixel intensity histogram, but they will sum to the total destructive interference Gaussian curve that was fit. The three fitted curves, f_{OO}, f_{DO} and f_{DD} can then be used to calculate expectation values of S for each pixel value. The expectation value of S at a given pixel, i, is equal to

$$\frac{{f}_{OO} ( i ) + 0.5 * {f}_{DO} ( i ) + 0 . * {f}_{D D} ( i )}{{f}_{OO} ( i ) + {f}_{DO} ( i ) + {f}_{D D} ( i )} ,$$

where the scaling factors are the S values corresponding to each of the underlying physical situations; S=1 for the case of pure constructive interference, S=0.5 for destructive interference from an ordered and disorder portion of the system, and S=0 for destructive interference from two disordered portions of the system. The expectation value of S can be squared and then for each pixel intensity can then be used to assign S² values to each pixel within the image by their corresponding pixel intensity value.

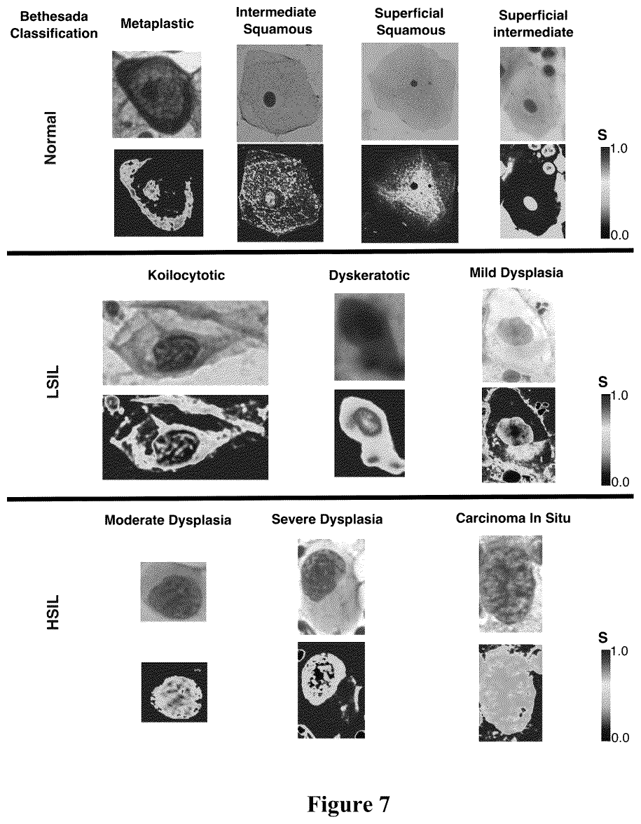

In terms of locations of order/disorder in the cell images themselves, FIG. 7 displays representative cells from each classification within the Herlev and SIPaKMeD databases, with their associated heat maps. In the heat map, the greatest level of disorder (blue) is observed within the cytoplasm of cells classified as normal. In low-grade cells, disorder is seen within the enlarged nucleus and irregular nuclear membrane correlation to coarse chromatin, which, when viewed in 3-D microscopy, appears to have “peaks and valleys”. In high-grade cells, the area of nuclear disorder is larger and denser.

Example 3. The same methodology may be applied across length scales, as demonstrated through an analysis of 530 images (120 cases) in the Multitask Cervical Histopathology (MTCH) dataset and 444 colposcopy images (85 cases) in the International Agency for Research on Cancer (IARC) database. They validate the observed S² ranges and further refined this into the lower anogenital squamous terminology (LAST) classifications as follows: benign (0.685-0.8613), CIN1 (0.5842-0.6329), CIN2 (0.5124-0.5481), CIN3 (0.3390-0.5022), and squamous cervical cancer (0.1283-0.2599). Diagnostic accuracy statistical tests demonstrated perfect precision for all categories in blind analyses when classifying a random sample of images using these ranges.

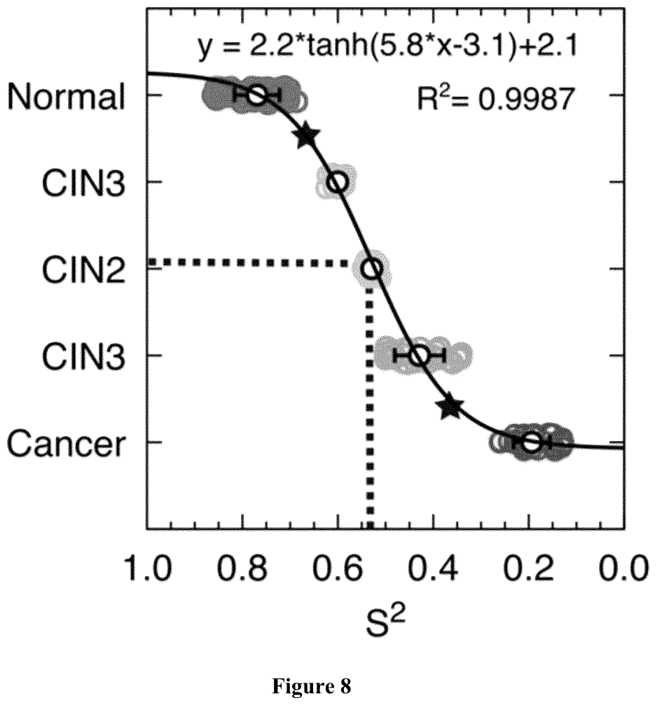

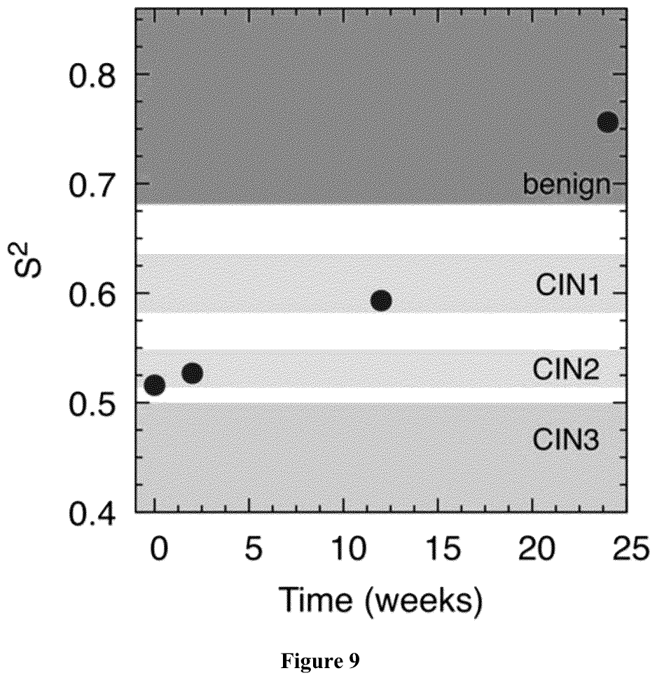

A regression analysis was undertaken of the relationship between observed S² values from the colposcopy images and the confirmed histology diagnoses from the IARC database. The fitted function indicates that non-linear changes occur when acquiring HPV and progressing to a pre-cancerous state, demonstrated by the star in FIG. 8. This finding correlates with the known biological function that HPV persistence does not progress sequentially from normal to CIN 1, then 2, and then 3. Furthermore, it suggests that once a critical threshold of HPV-induced activity is reached within a cell, the cell rapidly decreases in structural ordering (FIG. 8). Following this, a secondary analysis of time-lapsed cervical images was performed. An individual diagnosed with high-risk HPV and a low-grade squamous intraepithelial lesion was treated with a topical therapy and monitored with colposcopy exams at 2 weeks, 3 months, and 6 months, visually demonstrating regression of the cervical lesion. Similarly, order parameter measurements from these images demonstrated increased ordering with time (FIG. 9). The established ranges of order parameters associated with all classifications of cervical dysplasia, along with the evidence that changes in these parameters can indicate a resolving HPV infection, lead to the conclusion that temporal measurements of the order parameter yield critical insights into HPV carcinogenesis.

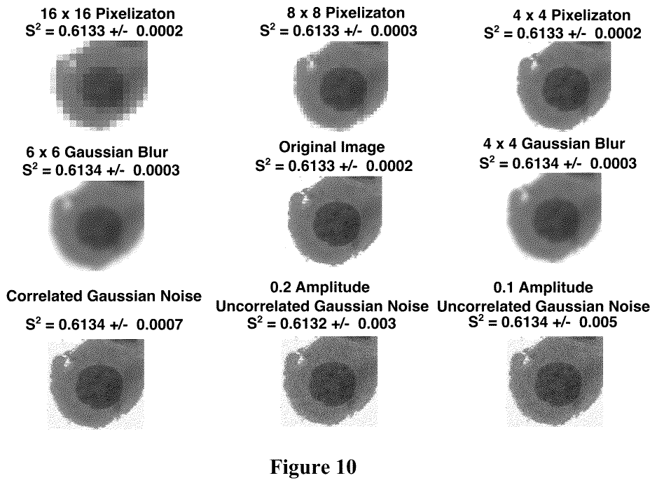

Example 4. The following example shows the method and systems' robustness to image quality. Our robustness analysis demonstrated that our technology is highly resilient to variations in image quality. FIG. 10 displays the results of artificial introduction of different types of noise into the images, including pixelation, Gaussian blur, correlated Gaussian noise, and uncorrelated Gaussian noise. Despite these distortions, our method consistently calculated the same order parameter from both the original and the noise-affected images.

This analysis of databases with large image archives has allowed us to assign an order number to a range of squamous cell abnormalities of the cervix. There is no overlap in order numbers between the classifications of normal, low grade or high grade when sorted into Bethesda classifications. The high diagnostic accuracy of our computer program with perfect sensitivity and specificity on this retrospective analysis suggests this may be a strong tool for clinicians to use. No other computational method has reached perfect accuracy, particularly for low grade and normal cytology results (Zhao et al. 2016; Gautam et al. 2018; Chankong et al. 2014; Zhang et al. 2017; Bora et al. 2017; Rahaman et al. 2021). Also, in contrast to other emerging technologies, such as deep learning and artificial intelligence, which often function as black boxes with qualitative interpretations, our method offers a clear, quantitative physical measure of malignancy. Lower degrees of ordering correspond to higher levels of cervical dysplasia, providing an easily interpretable result.

This technology is superior to existing artificial intelligence because it does not face the same limitations of 2-D analysis, uses quantitative rather than qualitative methodology and does not rely on binary classification of normal or abnormal and can provide a range of diagnoses. Several of these technologies may achieve high accuracy, but only for high-grade dysplasia. Our method is a quantitative physical measure of the cell, tissue or lesion under examination that provides an easily interpretable result with lower degrees of ordering corresponding to more severe cervical dysplasia. The picture quality of overlapping nuclei, debris or nontarget cells is felt to be less of a concern due to the analysis method of averaging ordered pixels above our threshold. This image analysis is not expected to require millions of observations for performance as is necessary for deep learning methodology and will require less configuring. It is also not prone to the same error of overfitting.

The order parameter-based image analysis method is implemented by a software program which, when configured accordingly, can be stored and executed on a storage medium or a data carrier that can be part of an analyzing unit such as a mobile phone or desktop computer. The nature of this method makes this technology adaptable to all forms of cervical cancer screening including pathologic analysis of cytology, histology or colposcopic images and is flexible to be applicable in both high and low resource settings. This program has the potential to work towards the World Health Organization's call to action for the elimination of cervical cancer by decreasing barriers to screening and treatment.

In some embodiments and aspects, a variety of sample preparation techniques (e.g., staining and slide preparation techniques) may be used a source for digital images for order analysis. Pre-existing photographs or slides may themselves be digitized for order analysis.

It will be understood by one having ordinary skill in the art that construction of the described device and other components is not limited to any specific material. Other exemplary embodiments of the device disclosed herein may be formed from a wide variety of materials, unless described otherwise herein.

It is also to be understood that variations and modifications can be made on the aforementioned structures and methods without departing from the concepts of the present disclosure, and further it is to be understood that such concepts are intended to be covered by the following claims unless these claims by their language expressly state otherwise.

The above description is considered that of the illustrated embodiments only. Modifications of the processes will occur to those skilled in the art and to those who make or use the processes. Therefore, it is understood that the embodiments shown in the drawings and described above are merely for illustrative purposes and not intended to limit the scope of the disclosure, which is defined by the following claims as interpreted according to the principles of patent law, including the Doctrine of Equivalents.

What is claimed is:

1. A computer-implemented method of diagnosing cervical cancer and pre cancer conditions in a patient utilizing an order parameter, the method comprising:
utilizing a computer to extract a numerical value corresponding to an order parameter squared (S²) from image data corresponding to at least one image of cervical tissue or cells from a patient, wherein the image data includes at least one region having light regions and dark regions, and wherein S² comprises a numerical value quantifying a degree of order present in the image data, and wherein the extracted numerical value comprises a ratio of an area of the light regions to a total area that is equal to the sum of an area of the light regions and an area of the dark regions;
comparing the extracted numerical value to one or more predefined diagnosis ranges corresponding to stages of cervical cancer and pre cancer conditions; and
diagnosing the stage of the cervical cancer and precancer conditions if the numerical value of the extracted S² falls within one of the predefined diagnosis ranges.

2. The method of claim 1, wherein:
the extracted numerical value comprises an extracted S² value; and including:
determining that the stage of the cervical cancer and precancer conditions is likely to be benign if the extracted S² value is greater than a predefined benign S² value, wherein the predefined benign S² value is the highest of one or more diagnosis ranges corresponding to stages of cervical cancer and pre cancer conditions.

3. The method of claim 2, further including:
determining that the stage of cervical cancer and precancer conditions is likely to be a precancer condition if the extracted S² value is within a predefined precancer S² value range, wherein the predefined precancer range is a value between the predefined benign S² range and the highest S² value of one or more diagnosis ranges corresponding to stages of cervical cancer.

4. The method of claim 3, including:
determining that the stage of cervical cancer and precancer conditions is likely to be a cervical cancer stage if the extracted S² value is below the predefined precancer S² value range.

5. The method of claim 1, wherein:
the one or more predefined diagnosis ranges corresponding to stages of cervical cancer and pre cancer conditions comprise one or more predefined S² value ranges that are determined by utilizing a computer to extract a numerical value of S² from a plurality of sets of data corresponding to images including images of cervical tissue or cells preclassified as one or more stages of cervical cancer and pre cancer conditions.

6. The method of claim 1, wherein:
extracting a numerical value corresponding to S² from image data includes selecting a region of interest that includes at least a portion of the at least one image of cervical tissue or cells.

7. The method of claim 6, wherein:
the image data includes at least some tissue or cells from the patient that are free of cervical cancer or precancer conditions; and
the region of interest does not include a portion of the least one image of cervical tissue or cells that is suspected to contain cervical cancer or pre cancer conditions.

8. The method of claim 6, wherein:
the image data comprises a plurality of pixels; and
extracting a numerical value corresponding to S² from the image data includes utilizing a computer to create a pixel intensity histogram of the region of interest.

9. The method of claim 8, wherein:
extracting a numerical value corresponding to S² from the image data includes utilizing a computer to fit first and second curves to the pixel intensity histogram corresponding to the light and dark regions, respectively.

10. The method of claim 9, wherein:
extracting a numerical value corresponding to S² from image data includes utilizing a computer to convert the image data to greyscale image data;
utilizing a computer to determine a threshold value of the pixel intensity between peaks of the first and second curves; and
utilizing a computer to perform a binary threshold on the region of interest using the threshold value to form a set of digital data corresponding to an image having only black and white pixels.

11. The method of claim 1, wherein:
the image data comprises a selected one of digital optical image data or image data from a microscope.

12. The method of claim 6, wherein:
selecting a region of interest comprises utilizing a computer to determine a border using an algorithm.

13. A computer-implemented method of distinguishing between benign and malignant skin conditions utilizing a numerical value determined from data corresponding to one or more images of cervical cancer or precancer cells or tissue, the method comprising:
utilizing a computer to extract a numerical value from image data corresponding to a digital image of cervical cancer or precancer cells or tissue, wherein the extracted numerical value comprises an extracted numerical value of an order parameter squared (S²), and wherein the digital image corresponding to the image data includes at least one region of concern comprising a potential cervical cancer or precancer condition cell or tissue having a total area including light regions and dark regions, and wherein the numerical value is determined, based at least in part, on an area of a selected one of the light regions and the dark regions to a total area, wherein the total area is equal to the sum of the areas of the light regions and the areas of the dark regions;
estimating the likelihood that the potential cervical cancer or precancer cell or tissue is cervical cancer or precancerous condition cell or tissue based, at least in part, on a comparison of the extracted numerical value to one or more predefined numerical criteria that take into account the likelihood that the cell or tissue is cervical cancer or precancerous.

14. The method of claim 13, wherein:
the numerical value is determined by dividing the area of the light regions by the total area.

15. The method of claim 13, including:
determining a threshold brightness value for the digital image;
determining the area of the light regions by summing the areas of the pixels having a brightness value above the threshold brightness value.

16. The method of claim 13, wherein:
the extracted numerical value comprises an extracted numerical value of an order parameter squared (S²).

17. The method of claim 13, wherein:
the extracted numerical value comprises an extracted S² value; and including:
determining that the stage of the cervical cancer and precancer conditions is likely to be benign if the extracted S² value is greater than a predefined benign S² value, wherein the predefined benign S² value is the highest of one or more diagnosis ranges corresponding to stages of cervical cancer and pre cancer conditions.

18. The method of claim 17, further including:
determining that the stage of cervical cancer and precancer conditions is likely to be a precancer condition if the extracted S² value is within a predefined precancer S² value range, wherein the predefined precancer range is a value between the predefined benign S² range and the highest S² value of one or more diagnosis ranges corresponding to stages of cervical cancer.

19. The method of claim 18, including:
determining that the stage of cervical cancer and precancer conditions is likely to be a cervical cancer stage if the extracted S² value is below the predefined precancer S² value range.
